(19) United States
(12) Patent Application Publication
(10) Pub. No.: US 2024/0398825 A1
(43) Pub. Date: Dec. 5, 2024

(54) MELOXICAM COMPOSITION, PHARMACEUTICAL PREPARATION, AND PREPARATION METHOD AND USE THEREOF

(71) Applicant: NANJING DELOVA BIOTECH CO. LTD., Nanjing (CN)

(72) Inventors: Qingsong WANG, Nanjing (CN); Wenliang ZHOU, Nanjing (CN); Qu WU, Nanjing (CN); Limin ZOU, Nanjing (CN)

(21) Appl. No.: 18/799,084

(22) Filed: Aug. 9, 2024

Related U.S. Application Data
(63) Continuation-in-part of application No. 17/044,767, filed on Oct. 1, 2020, now Pat. No. 12,059,423.

(30) Foreign Application Priority Data
May 11, 2018 (CN) 201810450456.6
May 11, 2018 (CN) 201810451076.4

(51) Int. Cl. A61K 31/5415; A61K 9/00; A61K 47/02; A61K 47/10; A61K 47/12; A61K 47/18

(52) U.S. Cl. CPC A61K 31/5415; A61K 9/0019; A61K 47/02; A61K 47/10; A61K 47/12; A61K 47/18

(57) ABSTRACT

A meloxicam composition, pharmaceutical preparation, preparation method and use thereof are provided. The composition enhances the solubility of meloxicam in liquid pharmaceutical preparation. The meloxicam composition provided herein is directly used for intravenous injection administration to quickly reach effective therapeutic concentration for post-operative analgesia. Furthermore, the preparation method involves stirring and dispersing in a short time at room temperature, and is scalable to the large-scale production.

### CROSS-REFERENCE TO RELATED APPLICATIONS

This application is a continuation-in-part of U.S. patent application Ser. No. 17/044,767, filed Oct. 1, 2020, which is a U.S. national entry of PCT International Application No. PCT/CN2019/086424, filed May 10, 2019, which claims the benefit of and priority to the prior Chinese patent applications: CN 201810450456.6 submitted to China National Intellectual Property Administration on May 11, 2018, which is entitled “Meloxicam composition, pharmaceutical preparation, and preparation method and use thereof”; and CN 201810451076.4 submitted to China National Intellectual Property Administration on May 11, 2018, which is entitled “Meloxicam composition comprising pH regulator, preparation method and use thereof”. All of the prior applications are hereby incorporated by reference in their entirety.

### TECHNICAL FIELD

The present disclosure belongs to the field of pharmaceutical preparation, specifically relates to a meloxicam composition, pharmaceutical preparation, preparation method and use thereof.

### BACKGROUND

Meloxicam (or “MLX”) is an enolic nonsteroidal anti-inflammatory drug having a molecular formula of C₁₄H₁₃N₃O₄S₂, a molecular weight of 351.40 and a chemical name of 4-hydroxy-2-methyl-N-(5-methyl-2-thiazolyl)-2H-1,2-benzothiazine-3-carboxamide-1,1-dioxide. The compound has the following chemical structural formula:

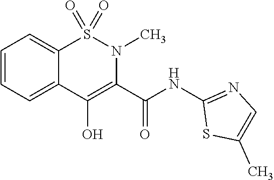 [SMILES: Cc1cnc(NC(=O)C2=C(O)c3ccccc3S(=O)(=O)N2C)s1]

Meloxicam is almost insoluble in water, its solubility in water is only 0.003414 mg/mL at 25° C., and it is slightly soluble in chloroform, acetone and ethanol. Therefore, although a variety of dosage forms of meloxicam have been developed by Boehringer Ingelheim Pharmaceuticals, Iroko Pharmaceuticals, Recro Pharma, etc., increasing the solubility of meloxicam is still a challenging subject in the development of intravenous injections of meloxicam.

For poorly soluble drugs, the physicochemical properties of the drugs can be changed to improve its solubility, such as by converting the drugs into salt forms or changing the crystal forms of the drugs. For example, Ochi et al. used the recrystallization method for converting meloxicam with Tris, arginine, diethanolamine (DEA), or triethanolamine (TEA), etc. into a salt form. Alladi Saritha et al. applied the spherical crystallization method for changing a crystal form of meloxicam into a spherical shape to increase its dissolution rate. However, the methods for forming salts or for changing crystal forms have limitations. For instance, it is required to separate drugs from their salts for the salt-forming methods, and the formation of a crystal is affected by various factors, such as stirring speed, stirring time, temperature and the type of solvent, etc., and limited because of the preparation methods and the stability of salts or crystal forms.

In addition, it is also possible to increase the solubility of poorly soluble drugs from the perspective of formulation, for example, by using mixed solvents, inclusion techniques or new techniques for new dosage forms. Currently, meloxicam has also been studied by the above-mentioned methods both domestic and abroad, but people confront with the problems of complicated preparation processes, excessive use of excipients, and a variety of excipients and organic solvents which are not suitable for intravenous injection administration.

For example, WO2008062274A2 discloses increasing the solubility of meloxicam by using a mixed solvent containing pyrrolidone, ethanol and water, but pyrrolidone is not suitable for intravenous administration. US2010137292A1 discloses solubilizing meloxicam by using a mixed solvent containing meglumine as an organic base, N,N-dimethylacetamide and polyethylene glycol, but N,N-dimethylacetamide is also not suitable for intravenous administration. Furthermore, CN103110575A discloses an ophthalmic preparation containing meloxicam at a concentration of 1 mg/mL prepared by using cyclodextrin as a solubilizer, and adding a stabilizer, a pH regulator, an antibacterial agent and an osmotic pressure regulator. US2017157061A1 discloses that the solubility of meloxicam is also increased by forming nanoparticles prepared with meloxicam and a stabilizer (comprising polyvinylpyrrolidone, a surfactant, etc.). U.S. Pat. No. 9,345,665B2 discloses a meloxicam nanoparticulate composition containing at least one surfactant and a stabilizer to improve drug loading of meloxicam. Woraphatphadung et al. disclose a chitosan micelle loaded meloxicam to increase the solubility of meloxicam. CN104825396A discloses particles with a particle size of greater than 1 μm by grinding meloxicam with a grinding matrix to improve the dissolution characteristics of the bioactive substance.

However, organic solvents, surfactants, cyclodextrins and other components were used too much in the above-mentioned methods, it is easy to cause safety problems for intravenous injections. Moreover, the preparation processes for new technologies of new dosage forms such as nanoparticles are more complicated, the risk of quality control is high, and there are certain difficulties in the commercial production.

The solubility of meloxicam in pure PEG 400 is 3.763 mg/mL, found by Neelam Seeder et al. If the solubility of meloxicam is up to 1.5 mg/mL, the solvent must be ethanol, propylene glycol, PEG 400, or a mixed solution of PEG 400 and ethanol (PEG 400≥40%), and the solution pH≥9.58. And if the solubility is up to 3.75 mg/mL, the solvent must be ethanol, propylene glycol, PEG 400, or a mixed solution of PEG 400 and ethanol (PEG 400≥80%), and the solution pH≥9.85. Zaira J. Cárdenas et al. found that the solubility of meloxicam in PEG 400 aqueous solution is increased with increasing the concentration of PEG 400, and the maximum solubility in pure PEG 400 is 9.2 mg/mL. Dattatray T. Modhave et al. reported that the meloxicam contains an amide bond in its molecule, which may undergo hydrolysis. Patent CN1236774C discloses a formulation containing organic bases, various cosolvents and surfactants, which can only be used for intramuscular injections due to limitations associated with the safety of excipients, and have to be prepared at 90° C., resulting in increasing the risk of degradation products, and harsh production conditions.

In summary, there is an urgent need to develop intravenous injections of meloxicam with improved safety and stability, and convenient preparation technique of formulations.

In another aspect, it is well known that when preparing meloxicam liquid preparations, especially injections, the issue of safety must be considered, for example, increasing the stability of preparations and decreasing the impurity content. However, it is usually a technical problem that perplexes technicians and difficult to be solved. Furthermore, because meloxicam is almost insoluble in water that the solubility in water is only 0.003414 mg/mL at 25° C., and meloxicam is slightly soluble in chloroform, acetone and ethanol, it is generally considered that such solubility properties are unfavorable to improve the stability of liquid-containing preparations. Therefore, under the premise of improving the stability and decreasing the impurity content, if the solubility of meloxicam liquid preparations can be further improved, it is beneficial to extend uses of meloxicam, improve patient compliance, and improve the safety and efficiency of the preparations.

However, for meloxicam injections, the solubility of poorly soluble drugs is usually improved by using mixed solvents or inclusion techniques. Currently, meloxicam has also been studied by the aforementioned methods both domestic and abroad, but people confront with the problems of complicated preparation processes, poor stability, and easily causing safety problems during intravenous injections, which seriously affect the quality control and safety of the injections.

Therefore, it is urgent to develop meloxicam liquid compositions with improved safety and stability, and decreased impurity content, or even with further enhanced solubility and convenient preparation technique of formulations.

### SUMMARY

According to an aspect of the present disclosure, the following technical solution is provided: a meloxicam composition containing meloxicam and cosolvent, wherein the cosolvent is mixed solvents containing water and an organic solvent.

According to an embodiment of the present disclosure, the concentration by volume of the organic solvent in the cosolvent is in the range of 1% to 80%, preferably 5% to 80%, such as 10% to 60%, or 15% to 50%, or 20% to 40%, for example, 15%, 18%, 19%, 20%, 25% or 30%.

According to the present disclosure, the organic solvent is one, two or more selected from ethanol, propylene glycol, butanediol, isopropanol, tetrahydrofurfurol, tetrahydrofuran polyethylene glycol ether, glycerol, dimethylacetamide, polyethylene glycol (also known as PEG, such as polyethylene glycol 300, polyethylene glycol 400, polyethylene glycol 600), etc.; preferably one, two or more selected from ethanol, propylene glycol, butanediol, tetrahydrofurfurol, glycerol, polyethylene glycol 300, polyethylene glycol 400; such as one, two or more selected from ethanol, propylene glycol, polyethylene glycol 300, polyethylene glycol 400; exemplarily, the organic solvent is selected from polyethylene glycol 300 and/or polyethylene glycol 400.

According to the present disclosure, water in the cosolvent can be that suitable for pharmaceutical purposes, such as purified water or water for injection.

According to the present disclosure, the pH value of the compositions is not particularly limited, and preferably selected to ensure that the compositions can be used as injection products, particularly intravenous injection products.

Preferably, the compositions are injection products, such as meloxicam intravenous injection products.

According to the embodiments of the present disclosure, the pH of the compositions can be in the range of 3.0 to 10.0, preferably 6.5 to 10.0, such as 6.8 to 9.0, or 7.2 to 9.0, particularly 7.4 to 9.0.

Alternatively, the compositions can also contain pH regulators.

According to the embodiments of the present disclosure, the pH regulator can be selected from an alkaline pH regulator and/or an acidic pH regulator, for example, those reagent that is used as a pH regulator suitable for injections or intravenous injections.

According to the embodiments of the present disclosure, the alkaline pH regulator can be one, two or more selected from sodium hydroxide, sodium carbonate, sodium bicarbonate, triethylamine, diethanolamine, sodium phosphate, disodium hydrogen phosphate, sodium dihydrogen phosphate, Tris (tri(hydroxymethyl)aminomethane), arginine, lysine, histidine and glycine; preferably, the alkaline pH regulator is one, two or more selected from sodium hydroxide, sodium carbonate, sodium bicarbonate, disodium hydrogen phosphate, sodium dihydrogen phosphate, sodium phosphate, Tris, arginine, lysine, glycine and triethylamine; more preferably, the alkaline pH regulator is selected from sodium hydroxide, sodium carbonate, sodium phosphate, Tris or lysine; for example, the alkaline pH regulator is selected from sodium hydroxide or lysine.

According to the embodiments of the present disclosure, the acidic pH regulator can be one, two or more selected from vitamin C (also known as ascorbic acid), lactic acid, malic acid, fumaric acid, citric acid, tartaric acid, succinic acid, hydrochloric acid, phosphoric acid and acetic acid; preferably, the acidic pH regulator is one, two or more selected from lactic acid, malic acid, tartaric acid, citric acid, hydrochloric acid, phosphoric acid and acetic acid.

According to the embodiments of the present disclosure, the ratio of the mass of meloxicam to the volume of the cosolvent can be 5 to 35 mg/mL, such as 7 to 32 mg/mL, or 10-30 mg/mL, 15-30 mg/mL, or 20-25 mg/mL, etc.

According to the embodiments of the present disclosure, the meloxicam composition contain meloxicam, a cosolvent and a pH regulator;
wherein the cosolvent is mixed solvents containing water and an organic solvent, in which the organic solvent is one, two or more selected from ethanol, propylene glycol, polyethylene glycol 300, and polyethylene glycol 400, preferably polyethylene glycol 300 and/or polyethylene glycol 400;
the volume concentration of the organic solvent in the cosolvent is in the range of 5% to 80%, such as 10% to 60%, or 15% to 50%, such as 20% to 40%, for example, 15%, 18%, 19%, 20%, 25% or 30%;
the pH regulator is selected from a alkaline pH regulator and/or an acidic pH regulator, in which the alkaline pH regulator is selected from sodium hydroxide, sodium carbonate, sodium phosphate, Tris or lysine, preferably sodium hydroxide or lysine; the acidic pH regulator is one, two or more selected from lactic acid, malic acid, tartaric acid, citric acid, hydrochloric acid, phosphoric acid and acetic acid;

The ratio of the mass of meloxicam to the volume of the cosolvent can be 5 to 35 mg/mL, such as 7 to 32 mg/mL, or 10-30 mg/mL, or 15-30 mg/mL, or 20-25 mg/mL, etc.

In the meloxicam compositions provided in the present disclosure, the amount of meloxicam is not particularly limited, for example, it may be up to 10 g or more, 50 g or more, 100 g or more, 200 g or more, or 300 g or more, and the specific amount thereof can be determined depending on the scale of production.

In the meloxicam compositions provided in the present disclosure, the amount of the cosolvent is not particularly limited, for example, up to 1 L or more, 5 L or more, 10 L or more, 15 L or more, or 20 L or more, and the specific amount thereof can be determined depending on the scale of production.

Preferably, meloxicam is dissolved in the cosolvent.

According to the embodiments of the present disclosure, the liquid meloxicam composition preferably is free of solubilizer and/or no surfactant; preferably, the compositions is free of solubilizer and surfactant. Wherein, the solubilizer comprises, but is not limited to, a solubilizer used in a prior pharmaceutical preparation, for example, a solubilizer known to be used for injections.

For example, the solubilizer comprises, but is not limited to, cyclodextrin and its derivatives, propyleneglycol, N,N-dimethylacetamide, dimethyl sulfoxide, povidone, macrogol 15 hydroxystearate, macrogol 12 hydroxystearate, glycofurol.

Preferably, the solubilizer comprises, but is not limited to, cyclodextrin and its derivatives, propyleneglycol, N,N-dimethylacetamide, dimethyl sulfoxide, povidone (also named as polyvinylpyrrolidone or PVP), macrogol 15 hydroxystearate, macrogol 12 hydroxystearate, glycofurol, phospholipid, cholic acid and its derivatives, polyoxyethylene sorbitan fatty acid esters, vitamin E polyethylene glycol succinate, sodium dodecyl sulfate, sodium dodecyl sulfonate, sodium octadecyl sulfate, sodium octadecyl fumarate, sodium tetradecyl sulfate, sodium hexadecadecyl sulfate.

Preferably, the solubilizer comprises, but is not limited to, cyclodextrin and its derivatives, propyleneglycol, N,N-dimethylacetamide, dimethyl sulfoxide, povidone, macrogol 15 hydroxystearate, macrogol 12 hydroxystearate, glycofurol, phospholipid, cholic acid and its derivatives, sorbitan esters, vitamin E polyethylene glycol succinate, sodium dodecyl sulfate, sodium dodecyl sulfonate, sodium octadecyl sulfate, sodium octadecyl fumarate, sodium tetradecyl sulfate, sodium hexadecadecyl sulfate, polyxylglycerides, polyoxyethylene stearates, polyoxyethylene laurates, polyoxyethylene monooleates, polyoxyethylene alkyl ethers.

Preferably, the solubilizer comprises, but is not limited to, cyclodextrin and its derivatives, propyleneglycol, N,N-dimethylacetamide, dimethyl sulfoxide, povidone, macrogol 15 hydroxystearate, macrogol 12 hydroxystearate, glycofurol, phospholipid, cholic acid and its derivatives, sorbitan esters, vitamin E polyethylene glycol succinate, sodium dodecyl sulfate, sodium dodecyl sulfonate, sodium octadecyl sulfate, sodium octadecyl fumarate, sodium tetradecyl sulfate, sodium hexadecadecyl sulfate, polyxylglycerides, polyoxyethylene stearates, polyoxyethylene laurates, polyoxyethylene monooleates, polyoxyethylene alkyl ethers, sucrose fatty acid esters, ethylene glycol fatty acid ester, propylene glycol fatty acid ester, castor oil, glycerol, sorbitol, mannitol, xylitol.

As an example, the cyclodextrin comprises α-cyclodextrin, β-cyclodextrin or γ-cyclodextrin, and the derivatives comprise, but are not limited to, one, two or more of an ether derivative, an ester derivative, polymer, etc. of α-cyclodextrin, of β-cyclodextrin, of γ-cyclodextrin; such as, the ether derivative is selected from one, two or more of a glucose derivative, a hydroxypropyl derivative, a methyl derivative, etc.; exemplarily, the derivative is selected from one, two or more of hydroxyethyl-β-cyclodextrin, hydroxypropyl-β-cyclodextrin, sulfobutyl-β-cyclodextrin, etc.

As an example, said povidone comprises, but is not limited to, PVP K12, PVP K15, PVP K17, PVP K25, PVP K30, PVP K60, and PVP K90.

As an example, phospholipid comprises egg yolk lecithin, soybean lecithin.

As an example, cholic acid and its derivatives comprise deoxycholic acid, sodium deoxycholate, taurine deoxycholic acid, ursodeoxycholic acid, chenodeoxycholic acid, glycocholic acid, sodium cholate, taurine deoxycholic acid sodium, ursodeoxycholic acid sodium, chenodeoxycholic acid sodium, glycocholic acid sodium.

As an example, sorbitan esters comprise sorbitan diisostearate, sorbitan dioleate, sorbitan monoisostearate, sorbitan monolaurate, sorbitan monooleate, sorbitan monopalmitate, sorbitan monostearate, sorbitan sesquiisostearate, sorbitan sesquioleate, sorbitan sesquistearate, sorbitan triisostearate, sorbitan trioleate, sorbitan tristearate, dehydrated sorbitol monolaurate, dehydrated sorbitol monopalmitate, dehydrated sorbitol monooleate, dehydrated sorbitol tristearate.

As an example, polyoxyethylene sorbitan fatty acid ester comprises Polysorbate 20, Polysorbate 40, Polysorbate 80, Polysorbate 65, Polysorbate 85, Polysorbate 60.

As an example, polyxylglyceride comprise Caprylocaproyl Polyoxylglycerides, Lauroyl Polyoxylglycerides, Linoleoyl Polyoxylglycerides, Oleoyl Polyoxylglycerides, Stearoyl Polyoxylglycerides.

As an example, Polyoxyethylene laurates comprise Polyoxyl 4 laurate, Polyoxyl 8 laurate, Polyoxyl 12 laurate, Polyoxyl 24 laurate, Polyoxyl 40 laurate, Polyoxyl 100 laurate, Polyoxyl 200 laurate, Polyoxyl 400 laurate, Polyoxyl 600 laurate, Polyoxyl 1000 laurate, Polyoxyl 6000 laurate.

As an example, Polyoxyethylene Monooleates comprise Polyoxyl 300 monooleate, Polyoxyl 400 monooleate, Polyoxyl 600 monooleate.

As an example, polyoxyethylene alkyl ethers comprises Polyoxyl 6 cetostearyl ether, Polyoxyl 20 cetostearyl ether, Polyoxyl 25 cetostearyl ether, Polyoxyl 2 cetyl ether, Polyoxyl 10 cetyl ether, Polyoxyl 20 cetyl ether, Polyoxyl 4 lauryl ether, Polyoxyl 9 lauryl ether, Polyoxyl 23 lauryl ether, Polyoxyl 2 oleyl ether, Polyoxyl 10 oleyl ether, Polyoxyl 20 oleyl ether, Polyoxyl 2 stearyl ether, Polyoxyl 10 stearyl ether, Polyoxyl 21 stearyl ether, Polyoxyl 100 stearyl ether.

As an example, sucrose fatty acid ester comprises sucrose monolaurate, sucrose monopalmitate.

As an example, ethylene glycol fatty acid ester comprises ethylene glycol monostearate, ethylene glycol monolaurate, ethylene glycol monooleate, ethylene glycol monopalmitate.

As an example, propylene glycol fatty acid ester comprises propylene glycol diethyl ester, propylene glycol monostearate, propylene glycol lauric acid monodiester, propylene glycol palmitic acid monodiester, propylene glycol oleic acid monodiester.
wherein, the surfactant comprises, but are not limited to, a surfactant known to be used for pharmaceutical preparations or compositions, for example, selected from a surfactant used for injections, such as one, two or more selected from polysorbate, a polyoxyethylene castor oil derivative, poloxamer, polyethylene glycol 15-hydroxystearate, etc.

Preferably, the surfactant comprises, but is not limited to, polysorbate, a polyoxyethylene castor oil derivative, poloxamer, polyethylene glycol 15-hydroxystearate, lauryl sarcosine, coconut sarcosine, nutmeg sarcosine, and stearyl sarcosine, lauric acid isopropanolamide, lauric acid diethanolamide, xylitol anhydride monooleate, xylitol anhydride monostearate, N,N-dimethylstearamide, N,N-dimethyllauramide, N,N-dimethyloleamide, N,N-dimethyloctyl decanamide, stearic acid, triolein, tripalmitin, tristearin, triacetin, tribehenin, glycerin monostearate, glyceryl monooleate, glycerol monocaprylate, glyceryl monoacetate, diethylene glycol monohexadecane-2, diethylene glycol monohexadecane-20.

As an example, polysorbate comprises polyoxyl 6 stearate, polyoxyl 8 stearate, polyoxyl 12 stearate, polyoxyl 20 stearate, polyoxyl 24 stearate, polyoxyl 40 stearate, polyoxyl 50 stearate, polyoxyl 100 stearate, polyoxyl 110 stearate.

As an example, polyoxyethylene castor oil derivative comprises polyoxyethylene (5) castor oil, polyoxyethylene (9) castor oil, polyoxyethylene (15) castor oil, polyoxyethylene (35) castor oil, polyoxyethylene (40) castor oil, polyoxyethylene (10) castor oil, polyoxyethylene (60) castor oil, polyoxyethylene (80) castor oil, polyoxyethylene (90) castor oil, polyoxyethylene (100) castor oil, polyoxyethylene (200) castor oil, polyoxyethylene (40) hydrogenated castor oil, Polyoxyethylene (10) hydrogenated castor oil, polyoxyethylene (60) hydrogenated castor oil, polyoxyethylene (30) hydrogenated castor oil, polyoxyethylene (50) hydrogenated castor oil, polyoxyethylene (100) hydrogenated castor oil, polyoxyethylene (200) hydrogenated castor oil.

As an example, poloxamer comprises poloxamer 188, poloxamer 124, poloxamer 237, poloxamer 338, poloxamer 407.

The present disclosure also provides a method for preparing the meloxicam compositions, comprising mixing meloxicam, water, an organic solvent, and optionally pH regulators, wherein the organic solvent is as defined above.

According to some embodiments of the present disclosure, the preparation method comprises mixing meloxicam and a cosolvent, wherein the cosolvent is mixed solvents containing water and an organic solvent.

According to the present disclosure, the preparation method can further comprise a step for providing the cosolvent, for example, mixing water and the cosolvent.

When the composition contains a pH regulator, the preparation method also comprises mixing water and the first pH regulator, and then further mixing with an organic solvent to obtain a mixed solution; or firstly mixing water and an organic solvent, and then further mixing with the pH regulator to obtain a mixed solution.

Preferably, the method of preparation also further comprises mixing the mixed solution and meloxicam to give a mixed solution containing meloxicam.

Preferably, the preparation method also comprises mixing the mixed solution containing meloxicam and the second pH regulator, wherein the first and the second pH regulators are the same or different, and are each independently selected from the above-defined pH regulators. Preferably, the first pH regulator is an alkaline regulator, and the second pH regulator is an acidic regulator.

In other embodiments of the present disclosure, the preparation method may comprise mixing an alkaline aqueous solution containing meloxicam with an organic solvent.

Preferably, the preparation method also comprises providing an alkaline aqueous solution containing meloxicam by firstly mixing the first pH regulator and water, and then mixing with meloxicam to obtain an alkaline aqueous solution containing meloxicam.

Preferably, the preparation method also comprises mixing the alkaline aqueous solution containing meloxicam and an organic solvent to obtain a mixed solution containing meloxicam, and then mixing with the second pH regulator, wherein the first and the second pH regulators are the same or different, and are each independently selected from the above-defined pH regulators.

According to the embodiments of the present disclosure, the preparation method can comprise the following steps:
1) mixing a pH regulator and water so that the pH of water is ≥10.0, for example, pH≥11.0, or pH≥12.0, or pH≥12.5, or pH≥13.0, to obtain an alkaline aqueous solution;
2) mixing an organic solvent and the alkaline aqueous solution obtained in step 1) to obtain a mixed solution;
3) adding meloxicam into the mixed solution obtained in step 2) to obtain a mixed solution containing meloxicam;
4) adjusting the pH of the solution obtained in step 3) to obtain a meloxicam composition.

Alternatively, according to the embodiments of the present disclosure, the preparation method can comprise the following steps:
1) mixing a pH regulator and water so that the pH of water is ≥10.0, for example, pH≥11.0, or pH≥12.0, or pH≥12.5, or pH≥13.0, to obtain an alkaline aqueous solution;
2) mixing meloxicam and the alkaline aqueous solution obtained in step 1) to obtain an alkaline aqueous solution containing meloxicam;
3) adding an organic solvent into the mixed solution obtained in step 2) to obtain a mixed solution containing meloxicam;
4) adjusting the pH of the solution obtained in step 3) to obtain a meloxicam composition.

Generally, the temperature in the preparation method is not particularly limited. For example, the temperature in step 1), 2), 3) or 4) can be in the range of 5° C. to 80° C., and these temperatures can be adjusted in accordance with specific conditions according to the present disclosure. According to the exemplary preparation method of the present disclosure, without waiting for the above-mentioned alkaline aqueous solution to cool, an organic solvent may be added under stirring. Preferably, the temperatures in step 1), 2), 3) or 4) are in the range of 10° C. to 60° C., more preferably 20° C. to 60° C.; as an example, the temperature can be 20° C., 25° C., 30° C., 40° C., 50° C. or 60° C.

According to the embodiments of the present disclosure, the stirring in each step such as step 1), 2), 3) or 4) may promote mixing, and the stirring time can be 10 minutes or more, for example, 20 minutes, 30 minutes, 40 minutes, 50 minutes, 60 minutes or more in the above different embodiments.

The present disclosure also provides a liquid preparation, such as an injection, preferably an intravenous injection, the injection containing the above-mentioned meloxicam compositions.

According to the present disclosure, the injection can also comprise a container, such as an ampoule, a vial or a multi-dose container, containing the above-mentioned meloxicam composition.

According to the present disclosure, the injection may comprise a small-volume injection (20 mL or less, such as 1 mL, 2 mL, 3 mL, 4 mL, 5 mL, 6 mL, 7 mL, 8 mL, 9 mL, 10 mL, 15 mL, or 20 mL) and a large-volume injection (50 mL or more, such as 50 mL, 60 mL, 70 mL, 75 mL, 80 mL, 90 mL, 100 mL, 250 mL, 500 mL, etc.).

The present disclosure also provides a method for preparing the injection, comprising placing the meloxicam composition in a container; preferably, the composition is also sterilized before or after being placed in the container. The sterilization may be moist heat sterilization or sterilization by filtration.

The present disclosure also provides the use of the above-mentioned meloxicam compositions in the treatment of diseases (such as post-operative analgesia, rheumatoid arthritis, painful osteoarthritis, ankylosing spondylitis).

The present disclosure also provides the use of the above-mentioned meloxicam compositions in the preparation of drugs, especially for the treatment of post-operative analgesia, rheumatoid arthritis, painful osteoarthritis or ankylosing spondylitis.

Preferably, the drug is an injection product, especially an intravenous injection product.

In order to improve the above technical problems, the following technical solution is also provided in the second aspect of the present disclosure: a meloxicam composition, the composition containing meloxicam, a solvent, and a pH regulator, wherein the pH regulator comprises at least citric acid.

According to the present disclosure, the solvent can be selected from water, organic solvent, and cosolvent, wherein the cosolvent is mixed solvents of water and an organic solvent.

According to the embodiments of the present disclosure, the volume concentration of the organic solvent in the cosolvent is in the range of 1% to 80%, preferably 5% to 80%, such as 10% to 60%, or 15% to 50%, or 15% to 25%, or 20% to 40%, for example, 15%, 16%, 17%, 18%, 19%, 20%, 25% or 30%.

According to the present disclosure, the organic solvent is one, two or more selected from ethanol, propylene glycol, butanediol, isopropanol, tetrahydrofurfurol, tetrahydrofuran polyethylene glycol ether, glycerol, polyethylene glycol (also known as PEG, such as polyethylene glycol 300, polyethylene glycol 400, polyethylene glycol 600), etc.; preferably one, two or more selected from ethanol, propylene glycol, butanediol, tetrahydrofurfurol, glycerol, polyethylene glycol 300, polyethylene glycol 400; such as one, two or more selected from ethanol, propylene glycol, polyethylene glycol 300, polyethylene glycol 400; exemplarily, the organic solvent is selected from polyethylene glycol 300 and/or polyethylene glycol 400.

According to the present disclosure, water in the cosolvent can be that suitable for pharmaceutical purposes, such as purified water and water for injection.

According to the present disclosure, the pH values of the compositions are not particularly limited, and preferably selected to ensure that the compositions can be used for injections, particularly intravenous injections.

Preferably, the compositions are injection products, such as meloxicam intravenous injection products.

According to the embodiments of the present disclosure, the pH of the compositions can be in the range of 3.0 to 10.0, preferably 6.5 to 10.0, such as 6.8 to 9.0, or 7.2 to 9.0, or 7.4 to 9.0, particularly, 7.5, 7.6, 7.7, 7.8, 7.9, 8.0, 8.1, 8.2, 8.3, 8.4, 8.5, 8.6, 8.7, 8.8 or 8.9.

According to the embodiment of the present disclosure, the pH regulator is selected from an acidic pH regulator or a combination of an acidic pH regulator and an alkaline pH regulator, provided that the acidic pH regulator contains at least citric acid.

According to the embodiments of the present disclosure, the acidic pH regulator can be selected from citric acid, or a mixture of citric acid and one, two or more acids selected from Vitamin C (also known as ascorbic acid), lactic acid, malic acid, fumaric acid, citric acid, tartaric acid, succinic acid, hydrochloric acid, phosphoric acid and acetic acid.

According to the embodiments of the present disclosure, the alkaline pH regulator can be one, two or more selected from sodium hydroxide, sodium carbonate, sodium bicarbonate, triethylamine, diethanolamine, sodium phosphate, disodium hydrogen phosphate, sodium dihydrogen phosphate, Tris (tri(hydroxymethyl)aminomethane), meglumine, arginine, lysine, histidine and glycine; preferably, the alkaline pH regulator is one, two or more selected from sodium hydroxide, sodium carbonate, sodium bicarbonate, disodium hydrogen phosphate, sodium dihydrogen phosphate, sodium phosphate, Tris, meglumine, arginine, lysine, glycine and triethylamine; more preferably, the alkaline pH regulator is selected from sodium hydroxide, sodium carbonate, sodium phosphate, Tris or lysine; exemplarily, the alkaline pH regulator is selected from sodium hydroxide or lysine.

In an exemplary technical solution of the present disclosure, the pH regulator comprises sodium hydroxide and/or meglumine, and citric acid; or, the pH regulator consists of sodium hydroxide and citric acid, or consists of meglumine and citric acid.

According to the embodiments of the present disclosure, the ratios of the mass of meloxicam to the volume of the cosolvent can be 5 to 35 mg/mL, such as 7 to 32 mg/mL, for example, 10-30 mg/mL, 15-30 mg/mL, 20-25 mg/mL, etc.

Preferably, meloxicam is dissolved in the cosolvent.

According to the embodiments of the present disclosure, the liquid meloxicam compositions preferably contain no solubilizer and/or no surfactant; preferably, the compositions contain no solubilizer and no surfactant. Wherein, the solubilizer comprises, but is not limited to, a solubilizer used in a pharmaceutical preparation, for example, selected from a solubilizer known to be used for injections, such as cyclodextrin and its derivatives. As an example, the cyclodextrin comprises α-cyclodextrin, β-cyclodextrin or γ-cyclodextrin, and the derivatives comprise, but are not limited to, one, two or more of an ether derivative, an ester derivative, polymer, etc. of α-cyclodextrin, of β-cyclodextrin, or of γ-cyclodextrin; such as, the ether derivative is selected from one, two or more of a glucose derivative, a hydroxypropyl derivative, a methyl derivative, etc.; exemplarily, the derivative is selected from one, two or more of hydroxyethyl-β-cyclodextrin, hydroxypropyl-β-cyclodextrin, sulfobutyl-β-cyclodextrin, etc.; wherein, the surfactant comprises, but are not limited to, a surfactant known to be used for pharmaceutical preparations or compositions, for example, selected from a surfactant used for injections, such as one, two or more selected from polysorbate 80, a polyoxyethylene castor oil derivative, poloxamer, polyethylene glycol 15-hydroxystearate, etc.

The present disclosure also provides a composition substantially consisting of meloxicam, water, polyethylene glycol and a pH regulator.

The term “consisting essentially of” means the claim is limited to the components specified in the claim “and those that do not materially affect the basic and novel characteristic(s)” of the claimed invention. Alternatively, “consisting essentially of” may also means the elements recited in the claim is, based on weight or volume percentage, at least 95%, or 96%, or 97%, or 98%, or 99%, or 99.5% of the meloxicam composition.

The present disclosure also provides a method for preparing the meloxicam composition, comprising mixing meloxicam, a solvent (such as water and/or an organic solvent), and a pH regulator, wherein the solvent and the pH regulator are as defined above.

According to some embodiments of the present disclosure, the preparation method comprises mixing meloxicam and a cosolvent, wherein the cosolvent is mixed solvents containing water and an organic solvent.

According to the present disclosure, the preparation method can further comprise the steps for providing the cosolvent, for example, mixing water and the cosolvent.

When the composition contains a pH regulator, the preparation method also comprises firstly mixing water and the first pH regulator, and then further mixing with an organic solvent to obtain a mixed solution; or firstly mixing water and an organic solvent, and then further mixing with the second pH regulator to obtain a mixed solution.

Preferably, the preparation method also further comprises mixing the mixed solution and meloxicam to obtain a mixed solution containing meloxicam.

Preferably, the preparation method also comprises mixing the mixed solution containing meloxicam and the second pH regulator.

In other embodiments of the present disclosure, the preparation method can comprise mixing an alkaline aqueous solution containing meloxicam and an organic solvent.

Preferably, the preparation method also comprises providing an alkaline aqueous solution containing meloxicam, for example, firstly mixing the first pH regulator and water, and then mixing with meloxicam to obtain an alkaline aqueous solution containing meloxicam.

Preferably, the preparation method also comprises mixing the alkaline aqueous solution containing meloxicam and an organic solvent to obatin a mixed solution containing meloxicam, and then mixing with the second pH regulator.

According to the present disclosure, the first and the second pH regulators are the same or different, and are each independently selected from the above-defined pH regulators, provided that the second pH regulator is an acidic pH regulator containing citric acid. Preferably, the first pH regulator is an alkaline pH regulator, and the second pH regulator is an acidic pH regulator containing citric acid.

According to the embodiments of the present disclosure, the preparation method can comprise the following steps:
1) mixing the first pH regulator and water so that the pH of water is ≥10.0, for example, pH≥11.0, or pH≥12.0, or pH≥12.5, or pH≥13.0, to obtain an alkaline aqueous solution;
2) mixing an organic solvent and the alkaline aqueous solution obtained in step 1) to obtain a mixed solution;
3) adding meloxicam into the mixed solution obtained in step 2) to obtain a mixed solution containing meloxicam;
4) adjusting the pH of the solution obtained in step 3) by using the second pH regulator to obtain a meloxicam composition.

Alternatively, according to the embodiments of the present disclosure, the preparation method may comprise the following steps:
1) mixing the first pH regulator and water so that the pH of water is ≥10.0, for example, pH≥11.0, or pH≥12.0, or pH≥12.5, to obtain an alkaline aqueous solution;
2) mixing meloxicam and the alkaline aqueous solution obtained in step 1) to obtain an alkaline aqueous solution containing meloxicam;
3) adding an organic solvent into the mixed solution obtained in step 2) to obtain a mixed solution containing meloxicam;
4) adjusting the pH of the solution obtained in step 3) by using the second pH regulator to obtain a meloxicam composition.

Generally, the temperature in the preparation method is not particularly limited. For example, the temperature in step 1), 2), 3) or 4) can be in the range of 5° C. to 80° C., and these temperatures can be adjusted in accordance with specific conditions according to the present disclosure. According to the exemplary method of the preparation of the present disclosure, without waiting for the above-mentioned alkaline aqueous solution to cool, an organic solvent may be added under stirring. Preferably, the temperature in step 1), 2), 3) or 4) is in the range of 10° C. to 60° C., more preferably 20° C. to 60° C.; as an example, the temperature can be 20° C., 25° C., 30° C., 40° C., 50° C. or 60° C.

According to the embodiments of the present disclosure, the stirring in each step such as step 1), 2), 3) or 4) can promote mixing, and the mixing time can be 10 minutes or more, for example, 20 minutes, 30 minutes, 40 minutes, 50 minutes, 60 minutes or more in the above different embodiments.

The present disclosure also provides a liquid pharmaceutical preparation, such as an injection, preferably an intravenous injection, and the injection containing the above-mentioned meloxicam compositions.

According to the present disclosure, the injection can also comprise a container, such as an ampoule, a vial or a multi-dose container, containing the above-mentioned meloxicam composition.

According to the present disclosure, the injection can comprise a small-volume injection (20 mL or less, such as 1 mL, 2 mL, 3 mL, 4 mL, 5 mL, 6 mL, 7 mL, 8 mL, 9 mL, 10 mL, 15 mL, or 20 mL) and a large-volume injection (50 mL or more, such as 50 mL, 60 mL, 70 mL, 75 mL, 80 mL, 90 mL, 100 mL, 250 mL, 500 mL, etc.).

The present disclosure also provides a method for preparing the injection, comprising placing a meloxicam composition in a container; preferably, the composition is also sterilized before or after being placed in the container. The sterilization can be moist heat sterilization or sterilization by filtration.

The present disclosure also provides a method for treating a disease, comprising administrating the composition or the liquid pharmaceutical injection according to the present disclosure to a subject, wherein the disease is selected from post-operative analgesia, rheumatoid arthritis, painful osteoarthritis and ankylosing spondylitis.

The present disclosure also provides an use of the above-mentioned meloxicam compositions in the treatment of a disease (such as post-operative analgesia, rheumatoid arthritis, painful osteoarthritis, or ankylosing spondylitis).

The present disclosure also provides an use of the above-mentioned meloxicam compositions in the preparation of a drug, especially for the treatment of post-operative analgesia, rheumatoid arthritis, painful osteoarthritis or ankylosing spondylitis.

Preferably, the drug is an injection product, particularly an intravenous injection product.

Preferably, the meloxicam composition also comprise pH buffers for maintaining pH stability. The pH buffers comprise, but are not limited to, one or more of phosphate buffers, citrate buffers, acetate buffers, carbonate buffers, amino acid buffers, tromethamine buffers, succinate buffers, maleate buffers, tartrate buffers.

Preferably, the meloxicam composition also comprise osmotic pressure regulators to ensure the osmotic pressure requirements for intravenous solutions. The osmotic pressure regulators comprise, but are not limited to, one or more of sodium chloride, mannitol, lactose, glucose, sorbitol, xylitol, sucrose, trehalose, glycerol.

Preferably, the meloxicam composition also comprise an antioxidant for maintaining chemical stability of the formulation. The antioxidant comprise, but are not limited to, one or more of ascorbic acid, ascorbic acid derivatives (such as ascorbyl palmitate, ascorbyl stearate, sodium ascorbate, calcium ascorbate, etc.), BHA (butylated hydroxyanisole), BHT (butylated hydroxytoluene), alkyl gallates, sodium metabisulfite, sodium bisulfite, sodium sulfite, sodium mercaptoethanesulfonate, sodium formaldehyde sulfoxylate, vitamin E and its derivatives (α-tocopherol, α-tocopherol acetate, α-tocopherol acetate, α-tocopherol succinate, β-tocopherol, γ-tocopherol, δ-tocopherol, and polyethylene glycol 1000 α-tocopheryl succinate), disodium EDTA, cysteine hydrochloride, methionine, thioglycerol, and sodium sulfite. The antioxidants account for about 0.01%-0.5%, preferably, the antioxidant account for about 0.05%-0.2%.

Preferably, the meloxicam composition contains trace amount of impurities, including meloxicam related compound B. Typically, the acceptance criteria of meloxicam related compound B is no more than 0.4%, the acceptance criteria of total impurities is no more than 1.0%.

Advantageous effects of the present disclosure at least comprise the following:

It is surprisingly found in the present disclosure that the pharmaceutical compositions provided in the first aspect of the present disclosure can significantly increase the solubility of meloxicam in liquid preparations. For example, when the pH of the solution systems is adjusted to 12.0 or more before the dissolution of meloxicam, and the organic solvent in the cosolvent is ≥5% by volume, the concentration of meloxicam may reach 10 mg/mL or even higher, which indicates that the solubility of the drug is greatly improved.

Meanwhile, it is also surprisingly found in the present disclosure that the composition of the present disclosure exhibit excellent stability. For example, when the organic solvent is in the same proportion in the cosolvent, and polyethylene glycol (such as PEG 300 or PEG 400, etc.) is used as one of the components in the cosolvent, the stability is significantly improved. No precipitation occurs during long-term storage, thereby effectively reducing the content of impurities such as degradation products, and stable liquid compositions are obtained under injection conditions (especially under intravenous injection conditions). In other words, the cosolvent selected in the present disclosure can dramatically increase the solubility of the drug, and also significantly enhance the stability of the drug.

Since excipients, such as solubilizers, surfactants, etc., are not contained in the compositions of the present disclosure, the irritability of the compositions tends to decrease and the safety is better. The meloxicam compositions of the present disclosure can even be directly used for intravenous injection bolus, and the effective therapeutic concentration for post-operative analgesia are quickly reached.

Furthermore, the preparation technique of the compositions is simple and it can be completed by stirring and dispersing in a short time at room temperature, and the large-scale production is easy to be achieved.

It is also surprisingly found in the present disclosure that the stability of the compositions provided in the second aspect of the present disclosure is significantly improved. In particular, when citric acid is used as an acidic pH regulator, the content of degradation product B is effectively reduced, thus dramatically improving the safety of the liquid preparations, and no precipitation occurs during long-term storage. Meanwhile, the solubility of meloxicam in the compositions of the present disclosure is significantly increased, and the concentration of meloxicam can reach 10 mg/mL or even higher. It is very beneficial to the use of meloxicam as an injection (especially as an intravenous injection).

Furthermore, the preparation technique of the compositions is simple and it can be completed by stirring and dispersing in a short time at room temperature, and the large-scale production is easy to be achieved.

### BRIEF DESCRIPTION OF THE DRAWINGS

FIG. 1 shows stirring compositions 1-6 in table B4 on day zero and day 15 both resulted in turbid solutions.

FIG. 2 shows the meloxicam composition according to the present disclosure is stable up to 28 months.

FIG. 3 shows meloxicam solubility in PEG solutions and PEG with different pH solution.

FIG. 4 shows meloxicam composition physical stability with different cosolvent.

### EXAMPLES

The present disclosure will be further described in more detail below in conjunction with specific examples. The following examples are merely illustrative of the present disclosure and are not to be construed as limiting the scope of the present disclosure. Any technology that is implemented based on the above-described contents of the present disclosure is encompassed within the scope of the present disclosure.

Unless otherwise specified, the active pharmaceutical ingredients and reagents used in the following examples are all commercially available, or can be prepared by known methods.

Wherein, the meloxicam content is 99.9% and the content of total impurities is 0.03% in raw materials in Examples A1 to A15 and Comparative Examples A1 to A5.

The method for determining the meloxicam content described in the examples and comparative examples of the present disclosure refers to the method in USP40-NF35 Monographs: Method for determination of meloxicam content.

### Example A1 Meloxicam Composition

### Formulation:

| | | |
|---|---|---|
| Formulation: Meloxicam | 1.0 | g |
| PEG 400 | 15 | mL |

| | |
|---|---|
| Water | added to 100 mL |
| Sodium hydroxide/phosphoric acid (pH regulator) | appropriate amount |

### Sodium Hydroxide/Phosphoric Acid (pH Regulator) Appropriate Amount

Preparation method: formulating a sodium hydroxide solution with a pH of 12.0, adding a formulation amount of PEG 400, stirring the mixture to be homogenous at room temperature, then adding meloxicam, stirring to dissolve meloxicam, and adjusting the pH of the solution to 7.8 with phosphoric acid to obtain the meloxicam composition. No insoluble particles/visible foreign matter occurred at room temperature. No insoluble particles/visible foreign matter occurred at 2-8° C. for 15 days.

### Example A2 Meloxicam Composition

### Formulation:

| | | |
|---|---|---|
| Formulation: Meloxicam | 1.0 | g |
| PEG 400 | 15 | mL |

| | |
|---|---|
| Water | added to 100 mL |
| Sodium hydroxide/phosphoric acid (pH regulator) | appropriate amount |

### Sodium Hydroxide/Phosphoric Acid (pH Regulator) Appropriate Amount

Preparation method: formulating a sodium hydroxide solution with a pH of 12.0, adding meloxicam, stirring the mixture to dissolve meloxicam at room temperature, then adding a formulation amount of PEG 400, stirring the mixture to be homogenous, and adjusting the pH of the solution to 7.4 with phosphoric acid to obtain the meloxicam composition. No insoluble particles/visible foreign matter occurred at room temperature. No insoluble particles/visible foreign matter occurred at 2-8° C. for 15 days.

### Example A3 Meloxicam Composition

### Formulation:

| | | |
|---|---|---|
| Formulation: Meloxicam | 1.0 | g |
| PEG 300 | 15 | mL |

| | |
|---|---|
| Water | added to 100 mL |
| Sodium hydroxide/phosphoric acid (pH regulator) | appropriate amount |

### Sodium Hydroxide/Phosphoric Acid (pH Regulator) Appropriate Amount

Preparation method: formulating a sodium hydroxide solution with a pH of 12.0, adding a formulation amount of PEG 300, stirring the mixture to be homogenous, then adding meloxicam, stirring to dissolve meloxicam, and adjusting the pH of the solution to 8.0 with phosphoric acid to obtain the meloxicam composition. No insoluble particles/visible foreign matter occurred at room temperature. No insoluble particles/visible foreign matter occurred at 2-8° C. for 15 days.

### Example A4 Meloxicam Composition

### Formulation:

| | | |
|---|---|---|
| Formulation: Meloxicam | 1.0 | g |
| PEG 400 | 15 | mL |

| | |
|---|---|
| Water | added to 100 mL |
| Sodium hydroxide/phosphoric acid (pH regulator) | appropriate amount |

### Sodium Hydroxide/Phosphoric Acid (pH Regulator) Appropriate Amount

Preparation method: formulating a sodium hydroxide solution with a pH of 12.0, adding a formulation amount of PEG 400, stirring the mixture to be homogenous, then adding meloxicam, stirring the mixture to dissolve the meloxicam, and adjusting the pH of the solution to 8.2 with phosphoric acid to obtain the meloxicam composition. No insoluble particles/visible foreign matter occurred at room temperature. No insoluble particles/visible foreign matter occurred at 2-8° C. for 15 days.

### Example A5 Meloxicam Composition

### Formulation:

| | | |
|---|---|---|
| Formulation: Meloxicam | 0.938 | g |
| PEG 400 | 10 | mL |

| | |
|---|---|
| Water | added to 50 mL |
| Sodium hydroxide/lactic acid (pH regulator) | appropriate amount |

### Sodium Hydroxide/Lactic Acid (pH Regulator) Appropriate Amount

Preparation method: formulating a sodium hydroxide solution with a pH of 12.0, adding a formulation amount of PEG 400, stirring the mixture to be homogenous, then adding meloxicam, stirring to dissolve meloxicam, and adjusting the pH of the solution to 7.8 with lactic acid to obtain the meloxicam composition. No insoluble particles/visible foreign matter occurred at room temperature. No insoluble particles/visible foreign matter occurred at 2-8° C. for 15 days.

### Example A6 Meloxicam Composition

### Formulation:

| | | |
|---|---|---|
| Formulation: Meloxicam | 0.938 | g |
| PEG 400 | 10 | mL |

| | |
|---|---|
| Water | added to 50 mL |
| Sodium hydroxide/malic acid (pH regulator) | appropriate amount |

### Sodium Hydroxide/Malic Acid (pH Regulator) Appropriate Amount

Preparation method: formulating a sodium hydroxide solution with a pH of 12.0, adding a formulation amount of PEG 400, stirring the mixture to be homogenous, then adding meloxicam, stirring to dissolve meloxicam, and adjusting the pH of the solution to 8.4 with malic acid to obtain the meloxicam composition. No insoluble particles/visible foreign matter occurred at room temperature. No insoluble particles/visible foreign matter occurred at 2-8° C. for 15 days.

### Example A7 Meloxicam Composition

### Formulation:

| | | |
|---|---|---|
| Formulation: Meloxicam | 0.938 | g |
| PEG 400 | 10 | mL |

| | |
|---|---|
| Water | added to 50 mL |
| Sodium hydroxide/ | appropriate amount |
| tartaric acid (pH regulator) | |

### Sodium Hydroxide/Tartaric Acid (pH Regulator) Appropriate Amount

Preparation method: formulating a sodium hydroxide solution with a pH of 12.0, adding a formulation amount of PEG 400, stirring the mixture to be homogenous, then adding meloxicam, stirring to dissolve meloxicam, and adjusting the pH of the solution to 8.2 with tartaric acid to obtain a meloxicam composition. No insoluble particles/visible foreign matter occurred at room temperature. No insoluble particles/visible foreign matter occurred at 2-8° C. for 15 days.

### Example A8 Meloxicam Composition

### Formulation:

| | | |
|---|---|---|
| Formulation: Meloxicam | 1.0 | g |
| Propylene glycol | 15 | mL |

| | |
|---|---|
| Water | added to 100 mL |
| Sodium hydroxide/ | appropriate amount |
| phosphoric acid (pH regulator) | |

### Sodium Hydroxide/Phosphoric Acid (pH Regulator) Appropriate Amount

Preparation method: formulating a sodium hydroxide solution with a pH of 12.0, adding a formulation amount of propylene glycol, stirring the mixture to be homogenous, then adding meloxicam, stirring to dissolve meloxicam, and adjusting the pH of the solution to 8.4 with phosphoric acid to obtain the meloxicam composition. No insoluble particles/visible foreign matter occurred at room temperature. A small number of insoluble particles occurred at 2-8° C. for 15 days.

### Example A9 Meloxicam Composition

### Formulation:

| | | |
|---|---|---|
| Formulation: Meloxicam | 1.0 | g |
| Glycerol | 15 | mL |

| | |
|---|---|
| Water | added to 100 mL |
| Sodium hydroxide/ | appropriate amount |
| phosphoric acid (pH regulator) | |

### Sodium Hydroxide/Phosphoric Acid (pH Regulator) Appropriate Amount

Preparation method: formulating a sodium hydroxide solution with a pH of 12.0, adding a formulation amount of glycerol, stirring the mixture to be homogenous, then adding meloxicam, stirring to dissolve meloxicam, and adjusting the pH of the solution to 8.6 with phosphoric acid to obtain the meloxicam composition. No insoluble particles/visible foreign matter occurred at room temperature. A small amount of insoluble particles occurred at 2-8° C. for 15 days.

### Example A10 Meloxicam Composition

### Formulation:

| | | |
|---|---|---|
| Formulation: Meloxicam | 1.0 | g |
| Butanediol | 15 | mL |

| | |
|---|---|
| Water | added to 100 mL |
| Sodium hydroxide/ | appropriate amount |
| phosphoric acid (pH regulator) | |

### Sodium Hydroxide/Phosphoric Acid (pH Regulator) Appropriate Amount

Preparation method: formulating a sodium hydroxide solution with a pH of 12.0, adding a formulation amount of butanediol, stirring the mixture to be homogenous, then adding meloxicam, stirring to dissolve meloxicam, and adjusting the pH of the solution to 8.2 with phosphoric acid to obtain a meloxicam composition. No insoluble particles/visible foreign matter occurred at room temperature. A small amount of insoluble particles occurred at 2-8° C. for 15 days.

### Example A11 Meloxicam Composition

### Formulation:

| | |
|---|---|
| Formulation: Meloxicam | 1.0 g or 1.5 g |
| PEG 400 | 15 mL |
| Sodium hydroxide/ | appropriate amount |
| citric acid (pH regulator) | |
| Water | added to 100 mL |

Preparation method: formulating a sodium hydroxide solution with a pH of 12.0, adding a formulation amount of PEG 400, stirring the mixture to be homogenous, then adding meloxicam, stirring to dissolve meloxicam, and adjusting the pH of the solution to 7.8, 8.0, 8.2, 8.4, 8.6, 8.8 or 9.0, respectively, with citric acid to obtain the meloxicam composition. No insoluble particles/visible foreign matter occurred at room temperature. No insoluble particles/visible foreign matter occurred at 2-8° C. for 15 days.

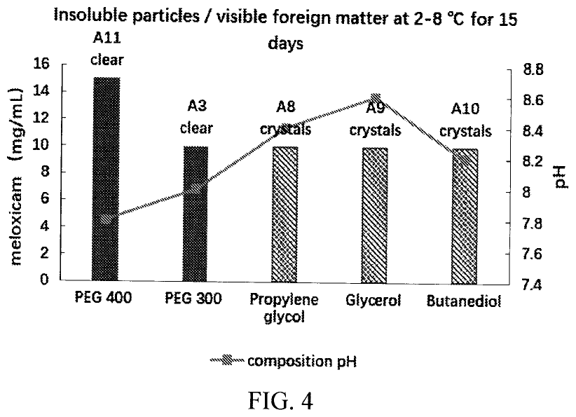

The physical stability of the meloxicam compositions with different cosolvents, such as A3, A8, A9, A10, and A11, is shown in FIG. 4.

### Example A12 Up-Scaling Preparation of the Meloxicam Compositions of the Present Disclosure

According to formulas F1 to F14 shown in the table below, 250 mL of liquid compositions with different components were prepared. Meloxicam compositions were obtained by formulating aqueous sodium hydroxide solutions with a pH of 13.0, adding a formulation amounts of organic solvent, stirring the mixtures to be homogenous, adding meloxicam, stirring to dissolve meloxicam, and adjusting the pH of the solutions to the corresponding values with phosphoric acid.

TABLE A1
Formulas of meloxicam liquid compositions

| | | PEG | PEG | Propylene | Purified | Measured |
|---|---|---|---|---|---|---|
| | MLX | 400 | 300 | glycol | water | pH of a |
| Sample | (g) | (mL) | (mL) | (mL) | (mL) | solution |
| F1 | 1.878 | 25 | / | / | 225 | 7.45 |
| F2 | 1.877 | 50 | / | / | 200 | 6.80 |
| F3 | 1.876 | 75 | / | / | 175 | 7.52 |
| F4 | 4.691 | 50 | / | / | 200 | 7.52 |
| F5 | 7.499 | 50 | / | / | 200 | 8.20 |
| F6 | 1.873 | 50 | / | / | 200 | 8.22 |
| F7 | 4.703 | 75 | / | / | 175 | 6.82 |
| F8 | 4.699 | 25 | / | / | 225 | 8.16 |
| F9 | 4.692 | 50 | / | / | 200 | 7.53 |
| F10 | 4.673 | 75 | / | / | 175 | 8.22 |
| F11 | 4.676 | / | 25 | / | 225 | 8.21 |
| F12 | 7.508 | / | 50 | / | 200 | 8.50 |
| F13 | 1.882 | / | / | 50 | 200 | 8.20 |
| F14 | 4.686 | / | / | 75 | 175 | 7.79 |

### Example A13 Stability Testing of Meloxicam Compositions

Each of the sample solutions of F1-F14 in Example A12 was sampled, filtered through 0.22 μm microporous filter membranes, and 5 mL of each filtrate was taken into 10 ml vials and the vials were sealed with caps. The vials were placed at room temperature, at 2-8° C. in the refrigerator, and at 60° C. in a constant temperature and humidity incubator, respectively, to investigate crystallization and changes of related substances. The results are shown in the table below.

TABLE A2
Stability testing of meloxicam compositions

| | Stability (30 days) | | | |
|---|---|---|---|---|
| | Insoluble | Insoluble | | |
| | particles/ | particles/ | Degradation | Degradation |
| | visible | visible | product B | product B |
| | foreign | foreign | content | content |
| | matter at | matter at | at 60° C. | at 25° C. |
| Sample | room temperature | 2-8° C. | (%) | (%) |
| F1 | No | No | 0.73 | 0.02 |
| F2 | No | No | 0.67 | 0.01 |
| F3 | No | No | 0.30 | 0.01 |
| F4 | No | No | 0.40 | 0.01 |
| F5 | No | No | 0.39 | 0.01 |
| F6 | No | No | 0.29 | 0 |
| F7 | No | No | 0.41 | 0.01 |
| F8 | No | No | 1.07 | 0.01 |
| F9 | No | No | 0.40 | 0.01 |
| F10 | No | No | 0.21 | 0.01 |
| F11 | No | No | no sample | no sample |
| F12 | No | No | 0.25 | no sample |
| F13 | No | occurring | no sample | no sample |
| | | a small | | |
| | | amount of | | |
| | | crystals | | |
| F14 | No | occurring | no sample | no sample |
| | | a small | | |
| | | amount of | | |
| | | crystals | | |

### Example A14 Up-Scaling Preparation of the Meloxicam Compositions of the Present Disclosure

Various liquid compositions with different formulas were prepared according to the formulas given in table A3. Aqueous sodium hydroxide solutions with a pH of 13.0 were prepared. The formulation amount of organic solvent was added. The mixtures were stirred to be homogenous. Different batches of meloxicam were added and dissolved with stirring. The pH of the solutions was adjusted to the corresponding pH with phosphoric acid. The solutions were sterilized to obtain meloxicam compositions.

TABLE A3
Formulas of meloxicam liquid compositions

| | | | Purified | Measured pH |
|---|---|---|---|---|
| Sample | MLX (g) | PEG 400 (L) | water (L) | of the solution |
| S1 | 10.0 | 0.2 | 0.800 | 8.6 |
| S2 | 15.0 | 0.2 | 0.800 | 8.8 |
| S3 | 300 | 3.54 | 15.62 | 8.55 |

### Example A15 Stability Testing of Meloxicam Compositions

The stability of the samples in Example A14 were investigated in long-term and accelerated conditions, and the results are shown in the table below.

Long-term stability testing: according to the guidelines on stability testing of drug substances and drug products in Chinese Pharmacopoeia 2015 edition, volume IV 9001, the long-term stability of samples S1 and S2 in Example A14 were investigated at a temperature of 30° C.±2° C., RH 65%±5%, for 3, 6, 9 and 12 months.

Accelerated stability testing: according to the guidelines on stability testing of drug substances and drug products in Chinese Pharmacopoeia 2015 edition, volume IV 9001, the accelerated stability of the samples S1, S2 and S3 in Example A14 were investigated at a temperature of 40° C.±2° C., RH 75%±5%, for 1, 2, 3 and 6 months.

TABLE A4
Stability testing of meloxicam compositions

| | | Stability | | |
|---|---|---|---|---|
| Sample | | S1 | S2 | S3 |
| 0 | Content (mg/g) | 9.5 | 14.2 | 14.8 |
| | Degradation | 0.13 | 0.12 | 0.00 |
| | impurity (%) | | | |
| | Total impurity (%) | 0.23 | 0.17 | 0.05 |
| | pH | 8.2 | 8.2 | 8.4 |
| Accelerated | Content (mg/g) | 9.5 | 14.3 | 14.8 |
| for 1 month | Degradation | 0.18 | 0.16 | 0.02 |
| (40° C.) | impurity (%) | | | |
| | Total impurity (%) | 0.24 | 0.22 | 0.21 |
| | pH | 8.5 | 8.5 | 8.5 |
| Accelerated | Content (mg/g) | / | / | 14.8 |
| for 2 months | Degradation | / | / | 0.03 |
| (40° C.) | impurity (%) | | | |
| | Total impurity (%) | / | / | 0.18 |
| | pH | / | / | 8.7 |
| Accelerated | Content (mg/g) | 9.5 | 14.2 | 14.9 |
| for 3 months | Degradation | 0.21 | 0.19 | 0.06 |
| (40° C.) | impurity (%) | | | |
| | Total impurity (%) | 0.33 | 0.31 | 0.21 |
| | pH | 8.7 | 8.7 | 8.5 |
| Long-term | Content (mg/g) | 9.5 | 14.3 | 14.8 |
| stability | Degradation | 0.14 | 0.14 | 0.02 |
| testing for | impurity (%) | | | |
| 3 months | Total impurity (%) | 0.25 | 0.25 | 0.07 |
| (30° C.) | pH | 8.6 | 8.7 | 8.8 |
| Accelerated | Content (mg/g) | 9.3 | 14.3 | / |
| for 6 months | Degradation | 0.31 | 0.28 | / |
| (40° C.) | impurity (%) | | | |
| | Total impurity (%) | 0.55 | 0.47 | / |
| | pH | 8.6 | 8.7 | / |
| Long-term | Content (mg/g) | 9.5 | 14.3 | / |
| stability | Degradation | 0.21 | 0.19 | / |
| testing for | impurity (%) | | | |
| 6 months | Total impurity (%) | 0.38 | 0.34 | / |
| (30° C.) | pH | 8.7 | 9.0 | / |
| Long-term | Content (mg/g) | 9.4 | 14.3 | / |
| stability | Degradation | 0.22 | 0.20 | / |
| testing for | impurity (%) | | | |
| 9 months | Total impurity (%) | 0.33 | 0.30 | / |
| (30° C.) | pH | 8.8 | 9.0 | / |
| Long-term | Content (mg/g) | 9.7 | 14.6 | / |
| stability | Degradation | 0.23 | 0.23 | / |
| testing for | impurity (%) | | | |
| 12 months | Total impurity (%) | 0.69 | 0.69 | / |
| (30° C.) | pH | 8.7 | 8.3 | / |

Note:
The 0-day test data was obtained immediately after sample preparation was completed.

The above testing results showed that the degradation impurities and total impurities could still be kept at low levels during long-term and accelerated stability testing of the meloxicam compositions prepared on a larger scale in Example A14, indicating that the meloxicam compositions exhibited excellent stability.

### Example A16 Meloxicam Composition

### Formulation:

| | |
|---|---|
| Meloxicam | 3.0 g |
| PEG 400 | 25 g/30 g/40 g/45 g |
| Sodium hydroxide/citric acid | appropriate amount |
| (pH regulator) | |
| Water | added to 100 mL |

Preparation method: formulating a sodium hydroxide solution with a pH of 12.0, adding four formulation amounts of PEG 400, stirring the mixture to be homogenous, then adding meloxicam, stirring to dissolve meloxicam, and adjusting the pH of the solution to 9.0±0.2, respectively, with citric acid to obtain the meloxicam composition. No insoluble particles/visible foreign matter occurred at room temperature. No insoluble particles/visible foreign matter occurred at 2-8° C. for 30 days. No insoluble particles/visible foreign matter occurred at 40° C. for 30 days. No insoluble particles/visible foreign matter occurred at 60° C. for 10 days.

### Example A17 Meloxicam Composition

### Formulation:

| | |
|---|---|
| Meloxicam | 3.0 g |
| PEG 400 | 30 g |
| Sodium hydroxide/acid | appropriate amount |
| (pH regulator) | |
| Water | added to 100 mL |

Preparation method: formulating a sodium hydroxide solution with a pH of 12.0, adding four formulation amounts of PEG 400, stirring the mixture to be homogenous, then adding meloxicam, stirring to dissolve meloxicam, and adjusting the pH of the solution to 7.6, 8.0 or 9.0, respectively, with acid (shown in the table A5) to obtain the meloxicam composition. No insoluble particles/visible foreign matter occurred at room temperature. No insoluble particles/visible foreign matter occurred at 2-8° C. for 30 days.

TABLE A5
meloxicam compositions

| | | Stability | |
|---|---|---|---|
| | | Insoluble | Insoluble |
| | | particles/visible | particles/ |
| | | foreign matter | visible foreign |
| Sample | | at room | matter at |
| | pH of the | temperature | 2-8° C. for |
| Acid Type | solution | for 30 days | 30 days |
| S1-citric acid | 7.6 | No | No |
| | 8.0 | No | No |
| | 9.0 | No | No |
| S2-phosphoric acid | 7.6 | No | No |
| | 8.0 | No | No |
| | 9.0 | No | No |
| S3-tartaric acid | 7.6 | No | No |
| | 8.0 | No | No |
| | 9.0 | No | No |
| S4-lactic acid | 7.6 | No | No |
| | 8.0 | No | No |
| | 9.0 | No | No |
| S5-maleic acid | 7.6 | No | No |
| | 8.0 | No | No |
| | 9.0 | No | No |
| S6-hydrochloric | 7.6 | No | No |
| acid | 8.0 | No | No |
| | 9.0 | No | No |
| S7-acetic acid | 7.6 | No | No |
| | 8.0 | No | No |
| | 9.0 | No | No |

### Comparative Example A1 Meloxicam Liquid Composition

### Formulation:

| | | |
|---|---|---|
| Formulation: Meloxicam | 0.938 | g |
| lysine | 0.730 | g |
| PEG 400 | 10 | mL |

| | |
|---|---|
| Water | added to 50 mL |
| phosphoric acid (pH regulator) | appropriate amount |

Preparation method: dissolving lysine and PEG 400 in water, stirring the mixture to be homogenous, adding meloxicam, stirring to dissolve meloxicam at 40° C., and adjusting the pH of the solution to 7.5 with phosphoric acid to obtain the meloxicam composition. No insoluble particles/visible foreign matter occurred at room temperature. No insoluble particles/visible foreign matter occurred at 2-8° C. for 15 days.

### Comparative Example A2 Meloxicam Liquid Composition

### Formulation:

| | | |
|---|---|---|
| Formulation: Meloxicam | 0.938 | g |
| meglumine | 0.975 | g |
| PEG 400 | 10 | mL |

| | |
|---|---|
| Water | added to 50 mL |
| phosphoric acid (pH regulator) | appropriate amount |

Preparation method: dissolving meglumine and PEG 400 in water, stirring the mixture to be homogenous, adding meloxicam, stirring for a long time above 40° C. to dissolve meloxicam, and adjusting the pH of the solution to 7.5 with phosphoric acid to obtain the meloxicam composition. No insoluble particles/visible foreign matter occurred at room temperature. No insoluble particles/visible foreign matter occurred at 2-8° C. for 15 days.

### Comparative Example A3 Meloxicam Liquid Composition

### Formulation:

| | | |
|---|---|---|
| Formulation: Meloxicam | 0.938 | g |
| arginine | 0.870 | g |
| PEG 400 | 10 | mL |

| | |
|---|---|
| Water | added to 50 mL |
| phosphoric acid (pH regulator) | appropriate amount |

Preparation method: dissolving arginine and PEG 400 in water, stirring the mixture to be homogenous, adding meloxicam, stirring for a long time above 40° C. to dissolve meloxicam, and adjusting the pH of the solution to 7.5 with phosphoric acid to obtain the meloxicam composition. A small amount of insoluble particles occurred at 2-8° C. for 7 days.

### Comparative Example A4

The solubility of meloxicam was investigated in an alkali or an alkaline amino acid solution (meglumine, Tris, arginine or lysine). The solubility is listed in the table below. The test results showed that the solubility of meloxicam in a high concentration organic base or basic amino acid could reach 10 mg/mL or more. However, it took a long time to complete the preparation (more than 6 hours) at room temperature, or meloxicam was dissolved at a higher temperature (40° C.). The preparation process was complicated. Crystals were precipitated after standing overnight at room temperature, and the solutions exhibited poor stability.

TABLE A5
Solubility of meloxicam in various concentrations
of organic bases or alkaline amino acids
Solubility (shaking at 25° C. for 24 h) (n = 3)

| | | | | | |
|---|---|---|---|---|---|
| Meglumine (mg/mL) | 5 | 10 | 15 | 20 | 30 |
| Solubility (mg/mL) | 8.01 | 17.83 | 24.63 | 32.03 | 44.97 |
| Tris (mg/mL) | 5 | 10 | 15 | 20 | 30 |
| Solubility (mg/mL) | 5.54 | 9.07 | 11.38 | 14.26 | 18.3 |
| Arginine (mg/mL) | 7 | 14 | 20 | 30 | 40 |
| Solubility (mg/mL) | 11.25 | 11.56 | 11.59 | 12.79 | 14.22 |
| Lysine (mg/mL) | 6 | 12 | 19 | 24 | 35 |
| Solubility (mg/mL) | 10.45 | 20.58 | 29.78 | 35.89 | 42.46 |

### Comparative Example A5

The solubility of meloxicam was investigated in PEG 400 aqueous solutions with varying ratios of PEG 400 to water. The solubility is shown in the table below. The results showed that the solubility of meloxicam in pure PEG 400 was only 2.27 mg/ml, which could not meet the requirements of intravenous injection.

TABLE A6
Solubility of meloxicam in PEG 400 aqueous solutions with
varying ratios of PEG 400 to water
Solubility (shaking at 25° C. for 24 h)

| PEG 400 | | | | | | | | | | | |
|---|---|---|---|---|---|---|---|---|---|---|---|
| (v/v %) | 0 | 10 | 20 | 30 | 40 | 50 | 60 | 70 | 80 | 90 | 100 |
| Solubility | 0.020 | 0.026 | 0.033 | 0.031 | 0.046 | 0.123 | 0.213 | 0.206 | 1.097 | 2.023 | 2.274 |
| (mg/mL) | | | | | | | | | | | |

### Solubility Study of Meloxicam in Injectable Solution

Preparation Method: Formulating a sodium hydroxide solution with pH of 12.0, adding a formulation amount of PEG 400, stirring the mixture to be homogeneous, then adding dual formulation amount of meloxicam, stirring until meloxicam dissolves, and adjusting the pH of the solution to 7.7-8.0 with citric acid to obtain the desired meloxicam composition.
The obtained meloxicam compositions were observed at room temperature and 2-8° C. respectively for two months. The compositions and liquid status thereof are detailed in Table A7.

TABLE A7
Prescription composition and liquid status

| | Meloxicam | PEG 400 | Dissolution | Liquid Status | |
|---|---|---|---|---|---|
| Number | mg/mL | mg/mL | Condition | Room Temperature 2-8° C. | |
| Solution | 30 | 300 | Dissolved | Clarified in 2 | Clarified in 2 |
| 1 | | | | months | months |
| Solution | 40 | 300 | Dissolved | Clarified in 2 | Clarified in 2 |
| 2 | | | | months | months |
| Solution | 50 | 300 | Dissolved | Clarified in 2 | Clarified in 2 |
| 3 | | | | months | months |
| Solution | 60 | 300 | Dissolved | Clarified in 2 | Clarified in 2 |
| 4 | | | | months | months |
| Solution | 70 | 300 | Slightly | / | / |
| 5 | | | Turbid | | |

Findings revealed that with PEG 400 usage at 300 mg/mL and active pharmaceutical ingredient (API) solubility at 60 mg/mL, the samples remained clear after two months' observation at both room temperature and 2-8° C., devoid of insoluble particles or visible foreign substances. This innovation demonstrates a meloxicam API solubility capacity reaching 60 mg/mL in the meloxicam injection solution.

The solubility of meloxicam was also investigated in PEG 200, PEG300 or PEG600, the solubility is shown in the table below.

TABLE A8
Solubility of meloxicam in PEG 200, PEG300 or PEG600

| | Solubility (shaking at 25° C. for 24 h) |
|---|---|
| Cosolvent | (mg/mL) |
| PEG200 | 3.60 |
| PEG300 | 2.81 |
| PEG600 | 3.18 |

The above test results showed that the compositions of the present disclosure, which contained polyethylene glycol such as PEG 400 or PEG200 or PEG 300 or PEG600 as an organic solvent, unexpectedly significantly increased the meloxicam content in the prepared compositions. Hence, liquid formulations of meloxicam, especially intravenous injections, have better application prospects and a wider range of applications, and dramatically improve therapy efficiency and patient compliance, etc. Moreover, this effect became more pronounced when the compositions with the same meloxicam content contained higher concentration of polyethylene glycol.

Furthermore, it is surprising that the compositions of the present disclosure also exhibited excellent stability, and not only no precipitation occurred after long standing, but also degradation products and total impurities were kept at low levels. In particular, when the proportion of an organic solvent in a cosolvent was the same, the meloxicam composition prepared with the cosolvent containing polyethylene glycol, especially PEG 400, exhibited good stability and no precipitation took place after long standing.

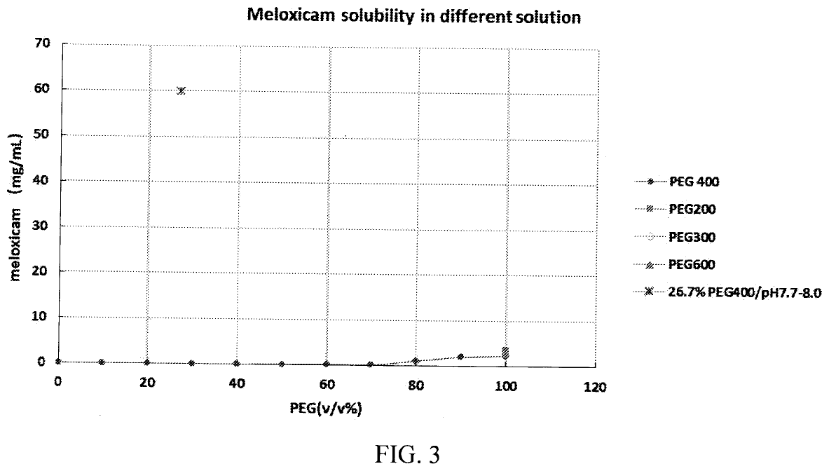

The solubility of meloxicam in PEG solutions and PEG with different pH solutions, such as 0%-100% PEG400 in Table A6, Solution 4 in Table A7, and 100% PEG200, PEG300, or PEG600 in Table A8, is shown in FIG. 3.

### Comparative Example A6 Meloxicam Liquid Composition

### Formulation:

| | |
|---|---|
| Meloxicam | 3.0 g |
| PEG 200 | 30 g |
| Sodium hydroxide/citric acid | appropriate |
| (pH regulator) | amount |
| Water | added to 100 mL |

Preparation method: formulating a sodium hydroxide solution with a pH of 12.0, adding a formulation amount of PEG 200, stirring the mixture to be homogenous, then adding a formulation amount of meloxicam, stirring to dissolve meloxicam, and adjusting the pH of the solution to 7.8-9.0 with citric acid to obtain the meloxicam composition. No insoluble particles/visible foreign matter occurred at room temperature. No insoluble particles/visible foreign matter occurred at 2-8° C. for 30 days.

### Comparative Example A7 Meloxicam Liquid Composition

### Formulation:

| | |
|---|---|
| Meloxicam | 3.0 g |
| PEG 300 | 30 g |
| Sodium hydroxide/citric acid | appropriate |
| (pH regulator) | amount |
| Water | added to 100 mL |

Preparation method: formulating a sodium hydroxide solution with a pH of 12.0, adding a formulation amount of PEG 300, stirring the mixture to be homogenous, then adding a formulation amount of meloxicam, stirring to dissolve meloxicam, and adjusting the pH of the solution to 7.8-9.0 with citric acid to obtain the meloxicam composition. No insoluble particles/visible foreign matter occurred at room temperature. No insoluble particles/visible foreign matter occurred at 2-8° C. for 30 days.

### Comparative Example A8 Meloxicam Liquid Composition

### Formulation:

| | |
|---|---|
| Meloxicam | 3.0 g |
| PEG 600 | 30 g |
| Sodium hydroxide/citric acid | appropriate |
| (pH regulator) | amount |
| Water | added to 100 mL |

Preparation method: formulating a sodium hydroxide solution with a pH of 12.0, adding a formulation amount of PEG 600, stirring the mixture to be homogenous, then adding a formulation amount of meloxicam, stirring to dissolve meloxicam, and adjusting the pH of the solution to 7.8-9.0 with citric acid to obtain the meloxicam composition. No insoluble particles/visible foreign matter occurred at room temperature. No insoluble particles/visible foreign matter occurred at 2-8° C. for 30 days.

In Examples B1-B10 and Comparative Examples B1-B4, the meloxicam content was 99.2% and the content of total impurities was 0.06% in the raw materials.

The “freeze-thaw” method was described as follows:

Placing the samples in a refrigerator at −20° C. for two days, then taking them out, and placing them under the accelerated condition of 40° C. for two days. Each freeze-thaw experiment was repeated three times, and the result at the end of the cycles was considered.

### Example B1 Meloxicam Liquid Composition

### Formulation:

| | |
|---|---|
| Meloxicam | 1 g or 1.5 g |
| PEG 400 | 15 mL |
| Sodium hydroxide/phosphoric acid | appropriate |
| or citric acid (pH regulator) | amount |
| Water | added to 100 mL |

Preparation method: formulating a sodium hydroxide solution with a pH of 12.0, adding a formulation amount of PEG 400, stirring the mixture to be homogenous, then adding two formulation amounts of meloxicam, stirring to dissolve meloxicam, and adjusting the pH of the solution to 7.8 with phosphoric acid or citric acid to obtain the meloxicam composition. No insoluble particles/visible foreign matter occurred at room temperature. No insoluble particles/visible foreign matter occurred at 2-8° C. for 15 days.

### Example B2 Stability Testing of Meloxicam Liquid Compositions

Each of the sample solutions prepared in Example B1 was sampled, filtered through 0.22 μm microporous filter membranes, and 5 mL of each filtrate was taken into 10 mL vials and the vials were sealed with caps. The vials were placed at 60° C. in a constant temperature and humidity incubator to investigate the increase of the related substances. The results are shown in Table B1.

TABLE B1
Stability testing of meloxicam compositions

| | | Degradation | Total impurity |
|---|---|---|---|
| | Time | product B | content |
| Sample | (day) | content (%) | (%) |
| Meloxicam 15 mg/mL | 0 | 0.01 | 0.05 |
| adjust pH with | 9 | 0.16 | 0.29 |
| phosphoric acid | | | |
| Meloxicam 10 mg/mL | 0 | 0.01 | 0.09 |
| adjust pH with | 9 | 0.23 | 0.39 |
| phosphoric acid | | | |
| Meloxicam 10 mg/mL | 0 | 0 | 0.09 |
| adjust pH with | 9 | 0.07 | 0.23 |
| citric acid | | | |
| Meloxicam 15 mg/mL | 0 | Not detected | 0.06 |
| adjust pH with | 9 | 0.07 | 0.19 |
| citric acid | | | |

The above test results showed that, comparing with other pH regulators, the stability of meloxicam in the compositions containing citric acid as an acidic pH regulator was significantly improved and the formation of degradation product B was effectively reduced.

Moreover, when PEG 400 in Example B1 was replaced with other organic solvents such as ethanol, propylene glycol, butanediol, isopropanol, glycerin, PEG 300, etc., the results also showed that the contents of the degradation impurity B and total impurities in the compositions containing citric acid as a pH regulator were effectively reduced.

### Example B3 Meloxicam Liquid Composition

### Formulation:

| | | |
|---|---|---|
| Meloxicam | 1.5 | g |
| PEG 400 | 20 | mL |

| | |
|---|---|
| Sodium hydroxide/citric acid | appropriate |
| (pH regulator) | amount |
| Water | added to 100 mL |

Preparation method: formulating a sodium hydroxide solution with a pH of 12.0, adding a formulation amount of PEG 400, stirring the mixture to be homogenous, then adding meloxicam, stirring to dissolve meloxicam, and adjusting the pH of the solution to the required value (pH=8.0, 8.2, 8.4, 8.6, 8.8, 9.0) with citric acid to obtain the meloxicam compositions. No insoluble particles/visible foreign matter occurred at room temperature. No insoluble particles/visible foreign matter occurred at 2-8° C. for 15 days.

### Example B4 Stability Testing of Meloxicam Liquid Compositions

Each of the sample solutions prepared in Example B3 was sampled, filtered through 0.22 μm microporous filter membranes, and 5 mL of each filtrate was taken into 10 mL vials and the vials were sealed with caps. The vials were placed at 60° C. in a constant temperature and humidity incubator for 10 days to investigate the increase of the related substances. The investigation result was that the content of degradation product B remained stable below 0.2%. It showed that in the compositions containing citric acid as a pH regulator, the content of degradation product B could be controlled in a low content range, even when the pH of the samples varied over a wide range. Therefore, the stability of meloxicam in the compositions and the safety of the preparations were significantly improved.

### Example B5

With reference to the preparation method of Example B1, a meloxicam composition was prepared: 10% PEG 400 by volume, 18.75 mg/mL of meloxicam, and meglumine (instead of NaOH) and citric acid as pH regulators to adjust the pH of the composition to 8.2. No insoluble particles/visible foreign matter occurred at room temperature. No insoluble particles/visible foreign matter occurred at 2-8° C. for 15 days. After the product was freeze-thawed once, though small particles occurred at the bottom, they were dissolved rapidly after shaking.

Moreover, when the above-mentioned meglumine as a alkaline pH regulator was replaced with sodium hydroxide, sodium carbonate, sodium bicarbonate, triethylamine, diethanolamine, sodium phosphate, disodium hydrogen sodium phosphate, dihydrogen phosphate, Tris (tri(hydroxymethyl)aminomethane), arginine, lysine, histidine or glycine, etc., no insoluble particles/visible foreign matter occurred, either.

### Example B6

With reference to the preparation method of Example B1, a meloxicam composition was prepared: 10% PEG 400 by volume, 18.75 mg/mL of meloxicam, and NaOH and citric acid as pH regulators to adjust the pH of the composition to 8.2. No insoluble particles/visible foreign matter occurred at room temperature. No insoluble particles/visible foreign matter occurred at 2-8° C. for 15 days. After the product was freeze-thawed once, no insoluble particles/visible foreign matter occurred.

### Example B7

### Formulation:

| | |
|---|---|
| Meloxicam | 0.375 g |
| PEG 400 | 3 g |
| Sodium hydroxide/hydrochloric | appropriate amount |
| acid (pH regulator) | |
| Water | added to 20 mL |

Preparation method: formulating a sodium hydroxide solution with a pH of 12.0, adding a formulation amount of PEG 400, stirring the mixture to be homogenous, then adding a formulation amount of meloxicam, stirring to dissolve meloxicam, and adjusting the pH of the solution to 7.8 with hydrochloric acid to obtain the meloxicam composition. No insoluble particles/visible foreign matter occurred at room temperature. No insoluble particles/visible foreign matter occurred at 2-8° C. for 6 days.

### Example B8

### Formulation:

| | |
|---|---|
| Meloxicam | 0.375 g |
| PEG 400 | 3 g |
| Sodium hydroxide/acetic acid | appropriate |
| (pH regulator) | amount |
| Water | added to 20 mL |

Preparation method: formulating a sodium hydroxide solution with a pH of 12.0, adding a formulation amount of PEG 400, stirring the mixture to be homogenous, then adding a formulation amount of meloxicam, stirring to dissolve meloxicam, and adjusting the pH of the solution to 8.2 with hydrochloric acid to obtain the meloxicam composition. No insoluble particles/visible foreign matter occurred at room temperature. No insoluble particles/visible foreign matter occurred at 2-8° C. for 6 days.

### Example B9

### Formulation:

| | |
|---|---|
| Meloxicam | 0.375 g |
| PEG 400 | 3 g |
| Sodium hydroxide/phosphoric acid | appropriate |
| (pH regulator) | amount |
| Water | added to 20 mL |

Preparation method: formulating a sodium hydroxide solution with a pH of 12.0, adding a formulation amount of PEG 400, stirring the mixture to be homogenous, then adding a formulation amount of meloxicam, stirring to dissolve meloxicam, and adjusting the pH of the solution to 8.2 with phosphoric acid to obtain the meloxicam composition. No insoluble particles/visible foreign matter occurred at room temperature. No insoluble particles/visible foreign matter occurred at 2-8° C. for 6 days.

### Example B10

### Formulation:

| | |
|---|---|
| Meloxicam | 0.375 g |
| PEG 400 | 3 g |
| Meglumine/phosphoric acid | appropriate |
| (pH regulator) | amount |
| Water | added to 20 mL |

Preparation method: adding a formulation amount of meglumine and a formulation amount of PEG 400 in the water, stirring the mixture to be homogenous, then adding a formulation amount of meloxicam, stirring to dissolve meloxicam, and adjusting the pH of the solution to 8.2 with phosphoric acid to obtain the meloxicam composition. No insoluble particles/visible foreign matter occurred at room temperature. No insoluble particles/visible foreign matter occurred at 2-8° C. for 6 days.

### Comparative Example B1

With reference to the preparation method of Example B1, a meloxicam composition was prepared: 10% PEG 400 by volume, 18.75 mg/mL of meloxicam, and NaOH and hydrochloric acid as pH regulators to adjust the pH of the composition to 8.2. Insoluble particles/visible foreign matter occurred in the composition at 2-8° C. for 1 days.

### Comparative Example B2

With reference to the preparation method of Example B1, a meloxicam composition was prepared: 10% PEG 400 by volume, 18.75 mg/mL of meloxicam, and NaOH and acetic acid as pH regulators to adjust the pH of the composition to 8.2. Insoluble particles/visible foreign matter occurred in the composition at 2-8° C. for 1 days.

### Comparative Example B3

With reference to the preparation method of Example B1, a meloxicam composition was prepared: 10% PEG 400 by volume, 18.75 mg/mL of meloxicam, and NaOH and phosphoric acid as pH regulators to adjust the pH of the composition to 8.2. Insoluble particles/visible foreign matter occurred in the composition at 2-8° C. for 6 days. After the product was freeze-thawed once, crystals occurred after standing, and were almost not dissolved after shaking.

### Comparative Example B4

With reference to the preparation method of Example B1, a meloxicam composition was prepared: 10% PEG 400 by volume, 18.75 mg/mL of meloxicam, and meglumine and phosphoric acid as pH regulators to adjust the pH of the composition to 8.2. No insoluble particles/visible foreign matter occurred at room temperature. Insoluble particles/visible foreign matter occurred at 2-8° C. for 10 days. After the product was freeze-thawed once, a large amount of fine solids were precipitated after standing, and were dissolved rapidly after shaking.

The above test results showed that, comparing with other pH regulators, the stability of meloxicam in the compositions containing citric acid as an acidic pH regulator was significantly improved, the formation of impurities was effectively reduced, and the safety and efficiency of the preparations were surprisingly improved.

### Comparative Example B5

U.S. 2010/0137292 A1 (“Turp”) discloses an aqueous EDTA free solution of meloxicam that contains: 20 mg/ml of meloxicam, 12.5 mg/ml of meglumine, 80 or 200 mg/ml of N,N-dimethyl acetamide, 200 or 400 mg/ml of propylene glycol, and 2.0 mg/ml povidone in Examples 1-3. The stability of the formulations disclosed in Turp were tested. The ingredients and the equipment are listed in Table B2 and Table B3.

TABLE B2

| Ingredients | Manufacturer | Batch No. |
|---|---|---|
| meloxicam | Shandong Xinhua Pharmaceutical | 21027 |
| | Co., Ltd. | |
| meglumine | Merck KGaA | TD16020843 |
| Povidone K17 (PVP | BASF SE | 91554556PO |
| K17) | | |
| dimethylacetamide | Sinopharm Chemical Reagent Co., | 20191015 |
| (DMA) | Ltd. | |
| Propylene glycol (PG) | Sinopharm Chemical Reagent Co., | 20170615 |
| | Ltd. | |
| Water for injections | Shiyao Yinhu Pharmaceutical Co., | 23121103056 |
| | Ltd. | |

TABLE B3

| Equipment | Model No. | Serial Number |
|---|---|---|
| electronic balance | BSA224S-CW | F2048 |

Compositions with and without povidone K-17 (PVP K17) were tested for stability. Table B4 shows the compositions that we tested. Compositions 1, 3, and 5 correspond to Examples 1, 2, and 3 in Turp, respectively. Compositions 2, 4, and 6 corresponds to Compositions 1, 3, and 5, respectively, except that Compositions 2, 4, and 6 do not contain PVP K17.

TABLE B4

| | Composition | Composition | Composition | Composition | Composition | Composition |
|---|---|---|---|---|---|---|
| | 1 | 2 | 3 | 4 | 5 | 6 |
| | 092701 | 092702 | 092703 | 092704 | 092705 | 092706 |
| | mg | Mg | mg | mg | mg | mg |
| Meloxicam | 400 | 400 | 400 | 400 | 400 | 400 |
| Meglumine | 250 | 250 | 250 | 250 | 250 | 250 |
| DMA | 1600 | 1600 | 1600 | 1600 | 600 | 600 |
| PG | 4000 | 4000 | 8000 | 8000 | 4000 | 4000 |
| PVP K17 | 40 | 0 | 40 | 0 | 40 | 0 |
| Water | Balance of 20 | Balance of 20 | Balance of 20 | Balance of 20 | Balance of 20 | Balance of 20 |
| | mL | mL | mL | mL | mL | mL |

Compositions in Table B4 were prepared according to the following procedure.
a. weigh prescribed amount of meglumine and PVP K17 and place them in a 20 mL volumetric flask;
b. add 5 ml of water, shake until solids dissolved;
c. add prescribed amount of DMA and PG, shake until uniformly mixed;
d. add prescribed amount of meloxicam, stir until fully dissolved;
e. add water until the solution volume reaches 20 mL;
f. filter the solution using 0.22 μm PVDF and placed in two 6 mL vials at 5 mL of solution each vial; and
g. place a first vial at room temperature and the second vial at 2-8° C., and observe for stability using a clarity detector.

The clarity of the compositions at day zero and day 15 are shown in Table B5.

TABLE B5

| | Batch | | Room Temp. 2-8° C. | |
|---|---|---|---|---|
| Name | Number | Day 0 | 15 days | 15 days |
| Composition 1 | 092701 | Clear | Clear | Forming floc |
| | | | | after shaking |
| Composition 2 | 092702 | Clear | Clear | Clear |
| Composition 3 | 092703 | Clear | Clear | Clear |
| Composition 4 | 092704 | Clear | Clear | Clear |
| Composition 5 | 092705 | Clear | Clear | Forming floc |
| | | | | after shaking |
| Composition 6 | 092706 | Clear | Forming floc | Forming floc |
| | | | after shaking | after shaking |

Solutions containing 30 mg/mL of meloxicam were tested for stability.

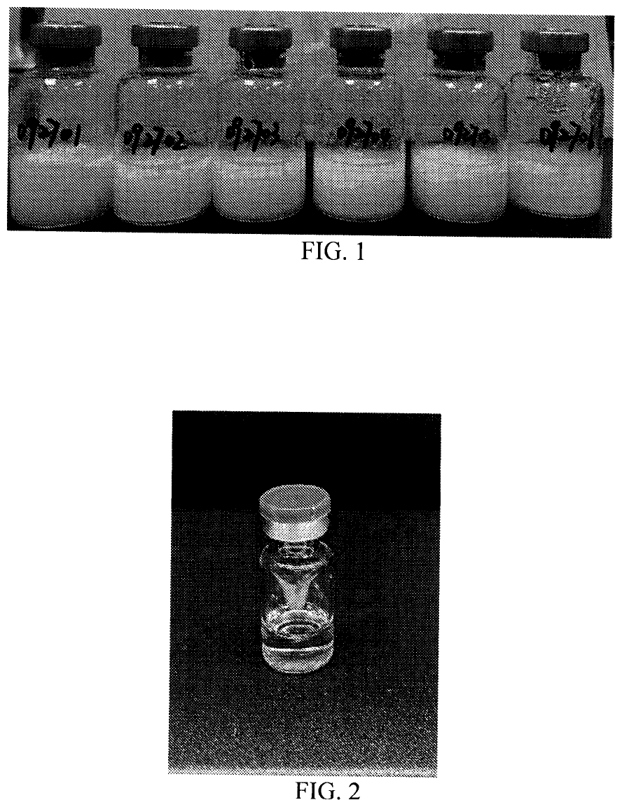

Meloxicam were added to 8 mL of each of compositions 1-6 until the meloxicam concentration reached 30 mg/mL. Stirring compositions 1-6 on day zero and day 15 both resulted in turbid solutions, shown in FIG. 1.

The data presented above indicates that, although 20 mg/mL meloxicam aqueous solution may be stable up to 15 days, 30 mg/mL meloxicam aqueous solutions were unstable on the same day they were prepared.

The data confirms that 30 mg/mL meloxicam solution is unstable, which provide a reason why there is no aqueous meloxicam solution available on the market.

The dramatic decrease in the shelf life meloxicam solution at 30 mg/mL demonstrates the difficulty in preparing a high concentration meloxicam aqueous solution.

Our meloxicam solution is stable up to 28 months, shown in FIG. 2.

The above embodiments of the present disclosure are described above. However, the present disclosure is not limited to the above embodiments. Any modification, equivalent alternative, improvement, etc., falling within the spirit and scope of the present disclosure, are intended to be comprised within the scope of the present disclosure.

1. A meloxicam composition, comprising meloxicam and a cosolvent, wherein the cosolvent is a mixed solvent comprising water and an organic solvent;
the organic solvent is selected from polyethylene glycols;
the volume concentration of the organic solvent in the cosolvent is in the range of 15% to 80%;
the meloxicam composition contains no solubilizer and no surfactant;
the pH of the composition is 6.5 to 10.0;
the composition is free of solubilizer and/or surfactant;
the solubilizer is selected from cyclodextrin and its derivatives, propyleneglycol, N,N-dimethylacetamide, dimethyl sulfoxide, povidone, macrogol 15 hydroxystearate, macrogol 12 hydroxystearate and glycofurol;
the surfactant is selected from, polysorbate, polyoxyethylated castor oil derivative, poloxamer, and 15-hydroxystearic acid-polyethylene glycol.

2. The composition according to claim 1, wherein:
the solubilizer is selected from cyclodextrin and its derivatives, propyleneglycol, N,N-dimethylacetamide, dimethyl sulfoxide, povidone, macrogol 15 hydroxystearate, macrogol 12 hydroxystearate, glycofurol, phospholipid, cholic acid and its derivatives, sorbitan esters, vitamin E polyethylene glycol succinate, sodium dodecyl sulfate, sodium dodecyl sulfonate, sodium octadecyl sulfate, sodium octadecyl fumarate, sodium tetradecyl sulfate, sodium hexadecadecyl sulfate, polyxylglycerides, polyoxyethylene stearates, polyoxyethylene laurates, polyoxyethylene monooleates, polyoxyethylene alkyl ethers, sucrose fatty acid esters, ethylene glycol fatty acid ester, propylene glycol fatty acid ester, castor oil, glycerol, sorbitol, mannitol, xylitol; and/or
the surfactant is selected from polysorbate, a polyoxyethylene castor oil derivative, poloxamer, polyethylene glycol 15-hydroxystearate, lauryl sarcosine, coconut sarcosine, nutmeg sarcosine, and stearyl sarcosine, lauric acid isopropanolamide, lauric acid diethanolamide, xylitol anhydride monooleate, xylitol anhydride monostearate, N,N-dimethylstearamide, N,N-dimethyllauramide, N,N-dimethyloleamide, N,N-dimethyloctyl decanamide, stearic acid, triolein, tripalmitin, tristearin, triacetin, tribehenin, glycerin monostearate, glyceryl monooleate, glycerol monocaprylate, glyceryl monoacetate, diethylene glycol monohexadecane-2, diethylene glycol monohexadecane-20.

3. The composition according to claim 1, wherein the organic solvent is selected from polyethylene glycol 300, polyethylene glycol 400, polyethylene glycol 600, and mixtures thereof.

4. The composition according to claim 1, wherein the pH of the composition is any of the following i) to v):
i) 6.8-10.0;
ii) 7.2-10.0;
iii) 7.2-9.0;
iv) 7.4-9.0; or
v) 7.4, 7.5, 7.6, 7.7, 7.8, 7.9, 8.0, 8.1, 8.2, 8.3, 8.4, 8.5, 8.6, 8.7, 8.8, 8.9, or 9.0.

5. The composition according to claim 1, wherein the cyclodextrin includes α-cyclodextrin, β-cyclodextrin, or γ-cyclodextrin;
the derivative includes an ether derivative, an ester derivative, or a polymer;
the ether derivative is one or more selected from glucose derivatives, hydroxypropyl derivatives, and methyl derivatives.

6. The meloxicam composition according to claim 1, wherein the volume concentration of the organic solvent in the latent solvent is one of the following i) to vii):
i) 15% to 60%,
ii) 15% to 50%,
iii) 15% to 40%,
iv) 20% to 40%,
v) 20% to 30%,
vi) 25% to 30%, or
vii) 15%, 16%, 17%, 18%, 19%, 20%, 25% or 30%.

7. The meloxicam composition according to claim 1, wherein the water in the latent solvent is any of the following i) to iii):
i) water suitable for pharmaceutical use;
ii) purified water, or
iii) Water for injection.

8. The meloxicam composition according to claim 1, wherein the mass-volume ratio of the meloxicam to the latent solvent is one of the following i) to iii):
i) 10-35 mg/mL,
ii) 15-35 mg/mL, or
iii) 20-35 mg/mL.

9. The meloxicam composition of claim 1, wherein the composition includes a pH adjusting agent.

10. The composition according to claim 9, wherein the pH regulator is selected from an alkaline pH regulator and/or an acidic pH regulator;
the alkaline pH regulator can be one, two or more selected from sodium hydroxide, sodium carbonate, sodium bicarbonate, triethylamine, diethanolamine, sodium phosphate, disodium hydrogen phosphate, sodium dihydrogen phosphate, Tris (tri(hydroxymethyl)aminomethane), arginine, lysine, histidine and glycine;
the acidic pH regulator can be one, two or more selected from ascorbic acid, lactic acid, malic acid, fumaric acid, citric acid, tartaric acid, succinic acid, hydrochloric acid, phosphoric acid and acetic acid.

11. The composition according to claim 9, wherein the pH adjuster is one of the following i) to iv):
i) at least citric acid;
ii) citric acid, or a mixture consisting of a combination of citric acid and one or more selected from ascorbic acid, lactic acid, malic acid, fumaric acid, tartaric acid, succinic acid, hydrochloric acid, phosphoric acid, and acetic acid;
iii) sodium hydroxide and/or meglumine, and citric acid;
iv) consisting of sodium hydroxide and citric acid, or meglumine and citric acid.

12. The composition according to claim 1, substantially consisting of meloxicam, water, polyethylene glycol and a pH regulator.

13. A method for preparing the composition according to claim 1, comprising mixing meloxicam, water, polyethylene glycol and optionally a pH regulator.

14. The method according to claim 13, comprising the following steps:
1) mixing the first pH regulator and water so that the pH of water is ≥10.0 to obtain an alkaline aqueous solution;
2) mixing polyethylene glycol and the alkaline aqueous solution obtained in step 1) to obtain a mixed solution;
3) adding meloxicam into the mixed solution obtained in step 2) to give a mixed solution containing meloxicam;
4) adjusting the pH of the solution obtained in step 3) by using the second pH regulator to obtain a meloxicam composition; or,
the method for preparing comprises the following steps:
1) mixing the first pH regulator and water so that the pH of water is ≥10.0, to obtain an alkaline aqueous solution;
2) mixing meloxicam and the alkaline aqueous solution obtained in step 1) to obtain an alkaline aqueous solution containing meloxicam;
3) adding polyethylene glycol into the mixed solution obtained in step 2) to obtain a mixed solution containing meloxicam;
4) adjusting the pH of the solution obtained in step 3) by using the second pH regulator to obtain a meloxicam composition;

15. The method according to claim 14, wherein 1) is any of the following i) to iii):
i) the first pH regulator is mixed with water to obtain an alkaline aqueous solution such that the pH value of the water is pH≥11.0;
ii) the first pH regulator is mixed with water to obtain an alkaline aqueous solution such that the pH value of the water is pH≥12.0; or
iii) the first pH regulator is mixed with water to obtain an alkaline aqueous solution such that the pH value of the water is pH≥12.5.

16. The method according to claim 14, wherein the first pH regulator is an alkaline regulator, and the second pH regulator is an acid regulator comprising at least citric acid.

17. A liquid pharmaceutical injection, comprising the composition according to claim 1.

18. The liquid pharmaceutical injection according to claim 17, wherein the injection is an intravenous injection.

19. A method for preparing the injection according to claim 17, comprising placing the composition in a container, wherein the composition is sterilized before or after being placed in the container.

20. A method for treating a disease, comprising administrating the composition according to claim 1 to a subject, wherein the disease is selected from post-operative analgesia, rheumatoid arthritis, painful osteoarthritis and ankylosing spondylitis.
